(19) United States
(12) Patent Application Publication
(10) Pub. No.: US 2018/0277244 A1
(43) Pub. Date: Sep. 27, 2018

(54) 3-TAP OR LESS PRACTICE MANAGEMENT SYSTEM

(71) Applicant: Praxify Technologies, Inc., Palo Alto, CA (US)

(72) Inventors: Abhijit Gupta, Pune (IN); Mohan Rao, Pune (IN)

(21) Appl. No.: 15/991,483

(22) Filed: May 29, 2018

Related U.S. Application Data
(63) Continuation of application No. PCT/IN2016/050421, filed on Nov. 28, 2016.

(30) Foreign Application Priority Data
Nov. 29, 2015 (IN) 4472/MUM/2015

(51) Int. Cl. G16H 10/60; G06F 3/0484; G16H 40/20

(52) U.S. Cl. CPC G16H 10/60; G16H 40/20; G06F 3/04842

(57) ABSTRACT

Methods, systems, and apparatus, including computer programs encoded on computer storage media for a 3-tap or less, electronic health record and practice management system. Implementations can include identifying an information item, an objective item, a decision item, an action item, and a context item each corresponding to a task in an electronic record and practice management environment. In response to identifying the information item, the object item, the decision item, the action item, and the context item, displaying an interactive mapping between the task and the information item, the object item, the decision item, the action item, and the context item on a display of a client device.

### CROSS-REFERENCE TO RELATED APPLICATIONS

This application is a continuation of and claims priority under 35 U.S.C. § 120 to PCT Application No. PCT/IN2016/050421, filed on Nov. 28, 2016, which claims priority to Indian Application No. 4472/MUM/2015, filed on Nov. 29, 2015, and each application is hereby incorporated by reference in its entirety.

### TECHNICAL FIELD

This specification generally relates to the field of information systems, computational systems, databases, networking systems, and communication systems.

### BACKGROUND

Medical practice entails activities in relation to human health and body, understanding and diagnosing various factors affecting human health and body, surgical procedures, examination procedures, diagnostic procedures, prognosis procedures, and the like activities. Qualified medical professionals can be equipped to deal with various facets of medical practice; in relation to the academic qualification that they have reached, and in relation to the professional experience that they have gained.

### SUMMARY

One general aspect of this subject matter relates to the field of healthcare information, healthcare technology, healthcare management, electronic medical records, electronic health records, decision support systems, and patient center care. Additionally, the subject matter relates to a 3-tap or less, electronic health record and practice management system.

The terms medical record, health record, encounter and medical chart can be used somewhat interchangeably to describe the systematic review and documentation of a single patient's medical or health journey that includes patient's history, diagnosis, prognosis, symptoms, vitals, review of systems, physical examination, medications, lab and diagnostics, allergies, surgical procedures and care across time not just within one particular health care provider setting, but also covering multiple health care providers and their interactions with the patient in context.

Medical records can include a variety of notes and data relating to doctor-patient interaction, doctor's interpretation of patient's complaints, diagnosis, prognosis, investigations and treatment plans. This data can include signs and symptoms data, review of various body systems data, examination data, vitals data, diagnosis data, medical decision making data, medical history data, family history, social history, previous surgical procedures and hospitalizations, any specific historical data of medicines taken, allergies, chronic and acute problems, lab reports, radiology images and reports' data, other investigation results' data, input/output data, drugs and immunization administration data and medication data, prognosis data, visit notes, insurance data, demographics, other relevant health histories, genomic data, data from wearables and other medical devices, and the like. Reviewing and maintenance of complete and accurate medical records is essential for the doctor as well as the patient for ensuing accurate diagnosis and treatment also from a general health and wellness perspective as well from a legal perspective.

Medical records can be used to understand the patient's current health status and past health history to ensure patient wellness and also to identify patient's diagnosis and provide/recommend relevant treatment protocol to a patient or fellow care providers for treating patients. Medical records can also be used as an aid to supplement the judgement and decision of a doctor/care provider. This is also a system with data of a patient captured at various stages of his/her life and used for a variety of medical and analytical purposes.

The types of personal health information that can be included in the medical records may cover the following: patient demographics information including but not limited to name, gender, birth date, blood type, race, ethnicity, marital status, address/geographical location and emergency contact information; a complete history of patients past visit histories; date of last physical exam; dates and results of tests and screenings; major illnesses and surgeries, with dates; a list of medicines, dosages and how long they can be being taken; any allergies and its reactions; any chronic/acute diseases and treatment plans; any history of illnesses in your family; dates and results of lab tests, imaging tests, and screenings; social history, family history; immunizations; risk assessments; care plans; vitals; data from wearables; genomic data; and, various clinical assessments and scores.

A care management system typically includes comprehensive medical records of patients and set of procedures and protocols that a doctor prescribes for a patient. In its electronic format, patient centered electronic medical record systems involve all the aspects of patient and illness/disease management, steps pertaining to which can be described above and may generally be referred during a patient-doctor interaction or for treating patients or for evolving better treatment protocols for future patients. For a doctor to review and record all aspects or facets of a patient, during the doctor-patient interaction, such patient centered electronic medical record systems be intuitive towards the workflows of that particular doctor and keep in context the various aspects of patient's demographic and medical information. These systems must provide patient medical information views to clinicians such that clinicians spend as little time as possible to find relevant information to ensure better diagnosis and medical decision making for treatment protocols and document important facets of the patient's encounter, and focus more on patient care as much as possible. Intuitiveness in this case means the ability of the system to understand how a clinician practices, learns from the clinician's practices, and be able to provide the right workflow, so that the clinician does not waste time in searching for information relevant in the context of the patient and his/her history and for documenting a patient record.

Typically, a patient is profiled in terms of demographics, medical history, family history, social history, current context (relating to season, epidemic, travel history, and the like), previous surgeries' history, investigations, vitals, current and previous problems, allergies, immunizations and the like.

In some implementations, one context of patient centered electronic medical record systems, a ‘doctor-patient interaction’ is meant to include the steps from understanding patient reported complaints, reviewing patient's medical records in the context of the complaints and condition, documenting history of present illnesses, to reviewing of body systems, to doing physical examination of the patient, to diagnosis to treatment plan to prognosis. In this context, the system can be used in correlation so the doctor-patient interaction is ready to ‘understand’ the interaction.

In some implementations, the system can be context-aware so that the system understands the doctor correctly in terms of pre-defined parameters.

In some implementations, the system can be context-aware so that the system understands the patient correctly in terms of pre-defined parameters.

In some implementations, the system can be context-aware so that the system understands the patient's previous records and histories correctly in terms of pre-defined parameters.

In some implementations, the system can be context-aware so that the system understands the doctor-patient interaction correctly in terms of pre-defined parameters.

In some implementations, the system can be context-aware so that the system understands the location and seasons correctly in terms of pre-defined parameters.

In some implementations, the system can be context-aware so that the system understands the demographic correctly in terms of pre-defined parameters.

In some implementations, the system can be context-aware so that the system understands how the doctor is interacting with the patient, how the doctor reviews medical records, and how the doctor documents in the patient centered electronic medical record system in terms of pre-defined parameters.

In some implementations, the system can be context-aware so that the system understands the current condition or state of the patient while documenting in the patient centered electronic medical record system in terms of pre-defined parameters.

In some implementations, the system can be context-aware so that the system understands which data set, for example, vitals and lab results, of the medical record should be promoted of the patient while reviewing patient medical records and documenting in the patient centered electronic medical record system in terms of pre-defined parameters.

In some implementations, the system can be context-aware so that it understands which actions, for example, adding a particular problem or suggesting a particular test, of the medical record should be promoted of the patient while documenting in the patient centered electronic medical record system in terms of pre-defined parameters.

The intelligent and intuitive system and method can be configured and designed so that a doctor is enabled and empowered to interact with the system in a context-aware manner. Therefore, the system and method context-aware and context-ready can be utilized so a doctor can interact with it.

With the advent of Internet of Things (IOT) and mobility, wearable devices and sensors have become ubiquitous in nature. Doctors today face a huge problem on understanding contexts from the trillions of bytes of information that they get from their patients. The vastness of this data needs to be interpreted by intelligent systems, and has to be presented to a doctor in a manner which will make logical sense for decision making. This intelligent system needs to be aware of various contexts in which these datasets were captured by these devices. Those contexts need to be interpreted in real time to aid the doctor to not take unnecessary interventions or measures, which will increase healthcare costs.

Also, each doctor has his/her own way of practicing and consuming patient data. The data needs to communicated to the doctor, what he/she is looking for answers to take real time decisions at point-of-care. This method of synthesizing data with various contexts and presenting to the doctor is one of the premises of this application. For example, a neurologist takes a round at an NICU to see a mid-aged male trauma patient, and sees his last 24 hours of vitals. The neurologist then reviews the last updated lab results, searches for a particular medication order, and sees what happened to vitals following the patient's use of that medication. The neurologist then looks for nursing notes to find any adverse events, and then goes to documenting the encounter. The machine can detect these types of patterns, and then the data can be synthesized for this doctor, for this type of patients with similar contexts and presented to the doctor to make quick and effective decisions.

For example, a 19 year old lady with a kid that is unmarried, has a history of abortion and abuse comes to the ER at a hospital, the system should automatically take into account risks associated and provide risk stratification in terms of suggesting to perform at least certain tests relating to abuse, abortion—etc.

The system should automatically pick up variations in the data or changes in the pattern of synthesis of data and compare those against what was expected and presents it upfront to the doctor to take actions, rather than the doctor figuring out the issue and treating reactively.

In general, one innovative aspect of the subject matter disclosed described in this specification can be embodied in methods that include the actions of identifying an information item corresponding to a task in an electronic record and practice management environment; identifying an objective item corresponding to the task in the electronic record and practice management environment; identifying a decision item corresponding to the task in the electronic record and practice management environment; identifying an action item corresponding to the task in the electronic record and practice management environment; identifying a context item corresponding to the task in the electronic record and practice management environment; in response to identifying the information item, the object item, the decision item, the action item, and the context item, displaying, by the one or more processors, an interactive mapping between the task and the information item, the object item, the decision item, the action item, and the context item on a display of a client device.

Other innovative aspects of the subject matter disclosed described in this specification can be embodied in methods that include: receiving, by the one or more processors, a first tap on the task on the display of the client device; and in response to the receiving the first tap, displaying, by the one or more processors, the information item, the decision item, the action item, and the context item in a linear arrangement on the display of the client device.

In some implementations, the method further includes receiving, by the one or more processors, a second tap on the task on the display of the client device; and in response to receiving the second tap, displaying, by the one or more processors, first contextual information corresponding to the information item, the decision item, the action item, and the context item on the display of the client device.

In some implementations, the method further includes receiving, by the one or more processors, a third tap on the task on the display of the client device; and in response to receiving the third tap, displaying, by the one or more processors, second contextual information corresponding to the first contextual information on the display of the client device.

In some implementations, the method further includes wherein the client device receives the first tap before the second tap, the first tap before the third tap, and the second tap before the third tap.

In some implementations, the method further includes receiving, by the one or more processors, weights to assign to the information item, the decision item, the action item, and the context item.

In some implementations, the method further includes wherein the task includes prescribing medicine to a patient from a doctor.

Other embodiments of this aspect include corresponding computer systems, apparatus, and computer programs recorded on one or more computer storage devices, each configured to perform the actions of the methods. A system of one or more computers can be configured to perform particular operations or actions by virtue of software, firmware, hardware, or any combination thereof installed on the system that in operation may cause the system to perform the actions. One or more computer programs can be configured to perform particular operations or actions by virtue of including instructions that, when executed by data processing apparatus, cause the apparatus to perform the actions.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 illustrates an example of a system of a mapping mechanism.

FIG. 2 illustrates a schematic of formation of a mapped item.

FIG. 3 illustrates various levels in accordance with a hierarchy defining mechanism.

Like reference numbers and designations in the various drawings indicate like elements.

### DETAILED DESCRIPTION

This specification seeks to provide a system and method for electronic medical and health records. The system and method for electronic medical and healthcare records can aid a doctor in identifying and quickly reviewing right medical information and it also aids the doctor to take decisions with a patient. Additionally, this system seeks to improve health care quality.

This specification seeks to provide a system and method for recording at least a facet of a patient and doctor interaction, such as during a patient doctor visit.

An additional object of the specification is to provide a system and method for providing a touch based, click based, voice based or gesture based recording of at least a facet of patient-doctor interaction/visit.

Yet an additional object of the specification is to provide an intuitive system and method for recording a patient-doctor interaction/visit.

Still an additional object of the specification is to provide a three or less haptic contact invoked visualization of a patient-doctor interaction/visit.

Another additional object of the specification is to provide a three or less haptic contact invoked recording of a patient-doctor interaction/visit.

An additional object of the specification is to provide a system and method, which is easy to use and understand for doctors as well as for patients, thereby increasing user adaptability.

Yet an additional object of the specification is to provide a system and method, which works in a similar visual and functional manner across all nodes, systems, and methods, such as across web interface portals, computer tablets, mobile phones, and smart watches.

Still an additional object of the specification is to provide a singular continuum of data access points, data flow, data related interfaces, data output points, data processing, data related parameters; across a plurality of devices, interfaces, nodes which can be associated, networked, or communicably coupled with this system and method.

Another additional object of the specification is to provide a singular behavior of data access points, data flow, data related interfaces, data output points, data processing, data related parameters; across a plurality of devices, interfaces, nodes which are associated, networked, or communicably coupled with this system and method.

Yet another additional object of the specification is to provide a singular form of data access points, data flow, data related interfaces, data output points, data processing, data related parameters; across a plurality of devices, interfaces, nodes which can be associated, networked, or communicably coupled with this system and method.

Still another additional object of the specification is to provide a singular function of data access points, data flow, data related interfaces, data output points, data processing, data related parameters; across a plurality of devices, interfaces, nodes which can be associated, networked, or communicably coupled with this system and method.

Yet another additional object of the specification is to provide a singular design of data access points, data flow, data related interfaces, data output points, data processing, data related parameters; across a plurality of devices, interfaces, nodes which can be associated, networked, or communicably coupled with this system and method.

This specification describes a 3-tap or less, electronic health record and practice management system. The system includes a mapping mechanism configured to map each task in a practice management system in terms of at least an initiator component and at least an impactor component. The system additionally includes at least an information identifier (II) in order to identify at least an information item that is a part of a task. The information identifier engaging with said mapping mechanism (MM) so that said task gets mapped in relation to at least an information item; at least an objectives identifier (OI) in order to identify at least an objective item that is a part of a task, said objective identifier engaging with said mapping mechanism (MM) so that said task gets mapped in relation to at least an objective item; at least a decision engine (DE) in order to identify at least a decision item that is a part of a task, said decision engine engaging with said mapping mechanism (MM) so that said task gets mapped in relation to at least a decision item; at least an action engine (AE) in order to identify at least an action item that is a part of a task, said mapping mechanism (MM)engaging with said action engine (AE) so that said task gets mapped in relation to at least an action item; at least a context engine (CE) in order to identify at least a context item that is a part of a task, said mapping mechanism (MM) engaging with said context engine (CE) so that said task gets mapped in relation to at least a context item; at least a node definition mechanism (NDM) in order to identify a relevant node item of a healthcare ecosystem, said mapping mechanism (MM) engaging with at least a node definition mechanism (NDM) thereby correlating a mapped item to at least one of an identified information item, an identified objective item, an identified decision item, an identified action item, an identified context item, an identified node item. At least a hierarchy defining mechanism (HDM) in order to define hierarchy (level) for each of said mapped items.

Typically, the hierarchy defining mechanism includes at least a first level defining mechanism (LDM 1) configured to define a first level of activity in respect of a mapped item.

Typically, the hierarchy defining mechanism includes at least a first level defining mechanism (LDM 1) configured to define a first level of activity in respect of a mapped item, characterized in that, the first level being a super node and in response to a first tap, a first level is activated.

Typically, the hierarchy defining mechanism includes at least a second level defining mechanism (LDM 2) configured to define a second level of activity in respect of an object to be achieved and which is pursuant to at least one of the first levels of activity.

Typically, the hierarchy defining mechanism includes at least a second level defining mechanism (LDM 2) configured to define a second level of activity in respect of an object to be achieved and which is pursuant to at least one of the first levels of activity, characterized in that, the second level being a child node below said super node, and in response to a second tap, a second level is activated.

Typically, the hierarchy defining mechanism includes at least a second level defining mechanism (LDM 2) configured to define a second level of activity in respect of an object to be achieved and which is pursuant to at least one of the first levels of activity, characterized in that, said second level being a child node below said super node, and in response to a second tap, a second level is activated, further characterized in that, in response to a first tap on an item at the first level, a user enters a corresponding second level, the second level including all actionable items contextual to the first level tapped action item.

Typically, the hierarchy defining mechanism includes at least a third level defining mechanism (LDM 3) configured to define a third level of activity in respect of an object to be achieved and which is pursuant to at least one of the second levels of activity.

Typically, the hierarchy defining mechanism includes at least a third level defining mechanism (LDM 3) configured to define a third level of activity in respect of an object to be achieved and which is pursuant to at least one of the second levels of activity, characterized in that, said third level being a sub node below said child node, and in response to a third tap, a third level is activated, further characterized in that, in response to a second tap on an item at the second level, a user enters a corresponding third level, the third level including all actionable items contextual to the second level tapped action item.

Typically, the system includes at least an intelligent action card (IAC) activated or authorized to carry out an action in relation to at least a mapped item (MI), said intelligent action card corresponding to at least a node in said system.

Typically, the system includes at least an intelligent action card (IAC) activated or authorized to carry out an action in relation to at least a mapped item (MI), said intelligent action card corresponding to at least a node in said system, characterized in that, said intelligent card being auto-generated and auto-presented at nodes in said system as defined by said system.

Typically, the system includes at least an intelligent action card (IAC) activated or authorized to carry out an action in relation to at least a mapped item (MI), characterized in that, said impactor component being embedded in said intelligent action card.

Typically, the system includes at least an intelligent action card (IAC) activated or authorized to carry out an action in relation to at least a mapped item (MI), characterized in that, the intelligent action card including at least one of an information item, an objective item, a decision item, an action item, a node item, or task-related items

Typically, the system includes at least an intelligent action card (IAC) activated or authorized to carry out an action in relation to at least a mapped item (MI), characterized in that, said intelligent action card being configured to apply a learned context in order to provide context-oriented intelligent action cards.

Typically, the system includes at least a customization mechanism configured to customize each task in relation to weights assigned to each node, thereby mapping each task in relation to at least a node item.

Typically, the system includes at least a customization mechanism configured to customize each task, logically, in terms of relevance.

For the purposes of this specification, the term, ‘doctor’ can include without limitations doctor, doctors, physicians, specialists, super specialists, dentists, surgeons, physiologists, psychiatrists, hospitalists, physiotherapists, medics, medical practitioners, medicos, nurses, nurse practitioners, physician assistants, paramedics, midwifes, clinical staff, and the likes of hospital related or healthcare related persons who deal with patients.

As discussed throughout this specification, a ‘tap’ is defined as a touch or a haptic contact or haptic engagement (whether discrete or continuous) or a click or a gesture, in response to which a pre-defined task or action takes place.

For the purposes of this specification, the term, ‘care management’, is meant to include actions, set of procedure and protocols adhered in a healthcare environment, which may include, but is not limited to scheduling, patient registration, patient onboarding, patient related document management, patient account management, billing, claims' processing, illness management, diagnosis, prognosis, examination, tests, results, interconnecting various nodes in the healthcare ecosystem, notifications and alarms, and the like.

According to this specification, there is provided a 3-tap or less, electronic health record and practice management system and method.

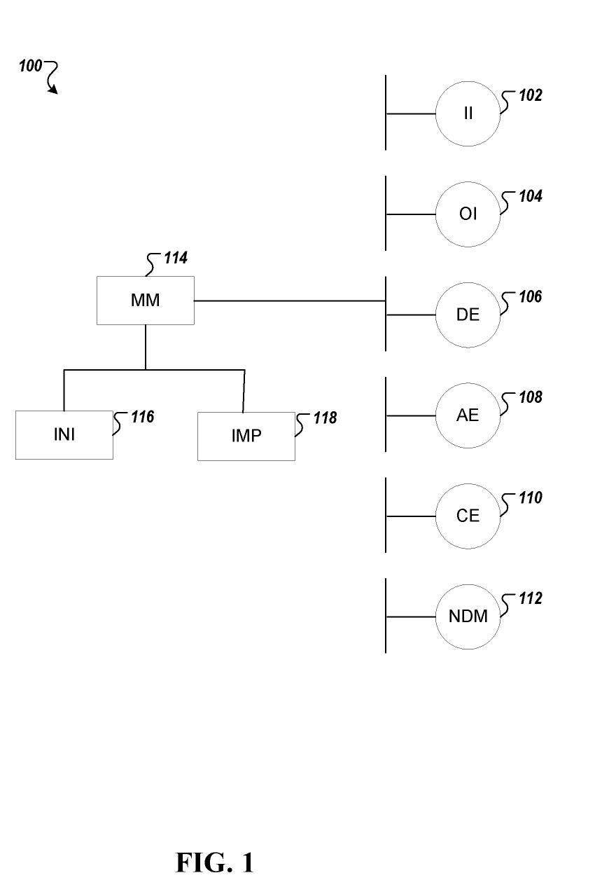

FIG. 1 illustrates a system 100 of a mapping mechanism. In some implementations, there is provided a mapping mechanism (MM) 114 to map each task in an electronic health record and practice management system and method in terms of at least an initiator (INI) 161 and at least an impactor (IMP) 118. According to the definition of the electronic health record and practice management system and method, a variety of tasks can be performed. Typically, these tasks are correlated, sequential, linear, dynamic, causal, or the like; depending upon the objective. However, in a healthcare ecosystem, protocols can be defined which correlate with the tasks. Considering these protocols, there is always a starting point and an ending point, which are to be mapped by this mapping mechanism in terms of at least an initiator and at least an impactor. These tasks can be identified in terms of information, objectives, decisions, actions, context, and/or nodes.

In some implementations, the mapping mechanism (MM) 114 engages with at least an information identifier (II) 102 in order to identify at least an information item that is a part of a task electronic health record and practice management system. Thus, a task can be mapped in relation to at least an information item. For example, an information item can be data related to a particular patient. The data relating to a particular patient can include any representation of that patient, such as his/her name, age, height, weight, previous illness, and medical history, to name a few examples. The task can be the doctor meeting with a patient at the NICU, such as a mid-aged male trauma patient, for example.

In some implementations, the mapping mechanism (MM) 114 engages with at least an objectives' identifier (OI) 104 in order to identity at least an objective item that is a part of a task of electronic health record and practice management system. Thus, a task can be mapped in relation to at least an objective item. Continuing with the example from above, the objective item can be data indicative of a task corresponding to the patient. The data indicative of a task include reviewing previous and current vital signs of the patient, reviewing lab results, searching for previous medication orders, and identifying one or more previous medical issues of the patient, for example.

In some implementations, the mapping mechanism (MM) 114 engages with at least a decision engine (DE) 106 in order to identify at least a decision item that is a part of an electronic health record and practice management system. Thus, a task can be mapped in relation to at least a decision item. Continuing with the example from above, the decision item can be data indicative of a decision that a doctor can make corresponding to the patient based on the information item and the objective item. The data indicative of a decision that a doctor can make can include identifying a type of medication or treatment to provide to the patient, for example.

In some implementations, the mapping mechanism (MM) 114 engages with at least an action engine (AE) 108 in order to identify at least an action item that is a part of an electronic health record and practice management system. Thus, a task can be mapped in relation to at least an action item. Continuing with the example from above, the action item can be data indicative of an action that a doctor can take in order to issue the decision item. For example, the data indicative of the action can include instructing the local pharmacy to fill the medication to provide to the patient.

In some implementations, the mapping mechanism (MM) 114 engages with at least a context engine (CE) 110 in order to identify at least a context item that is a part of an electronic health record and practice management system. Thus, a task can be mapped in relation to at least a context item to at least a node item. In at least one embodiment, a node may be any point in the healthcare ecosystem or the electronic health record and practice management system and method. Continuing with the example from above, the context item can data indicative of context from a patient that a doctor can recorder in the electronic health record and practice management system and method. For example, the context can include results for a how a patient is feeling in response to the prescribed treatment from the action item.

In some implementations, the mapping mechanism (MM) 114 engages with at least a node definition mechanism (NDM) 112 in order to identify a node item of a healthcare ecosystem or of an electronic health record and practice management system and method, which node is relevant. Weights may be assigned to the nodes in terms of relevance. Thus, a task can be mapped in relation to at least a node item. In at least one embodiment, a node may be any point in the healthcare ecosystem or the electronic health record and practice management system and method. For example, the node can identify a context item, an action item, a decision item, and an objective item.

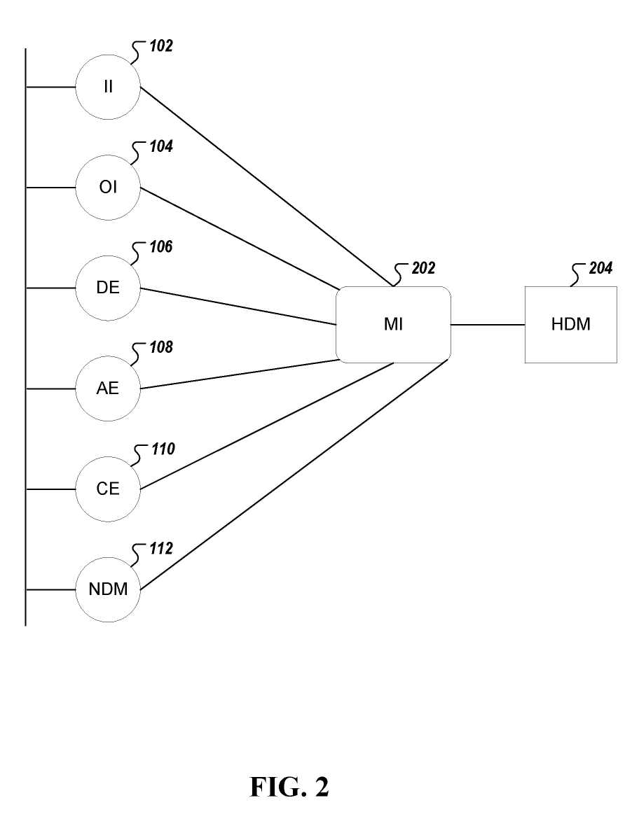

FIG. 2 illustrates a schematic of formation of a mapped item.

Therefore, a mapped item 202 is correlated to at least one of an identified information item 102, an identified objective item 104, an identified decision item 106, an identified action item 108, an identified context item 110, an identified node item 112, or the like identified items. In other words, a mapped item 202 is correlated to a defined task item of an electronic health record and practice management system and method. A hierarchy defining mechanism (HDM) may be used in order to define hierarchy (level) for each of the mapped items.

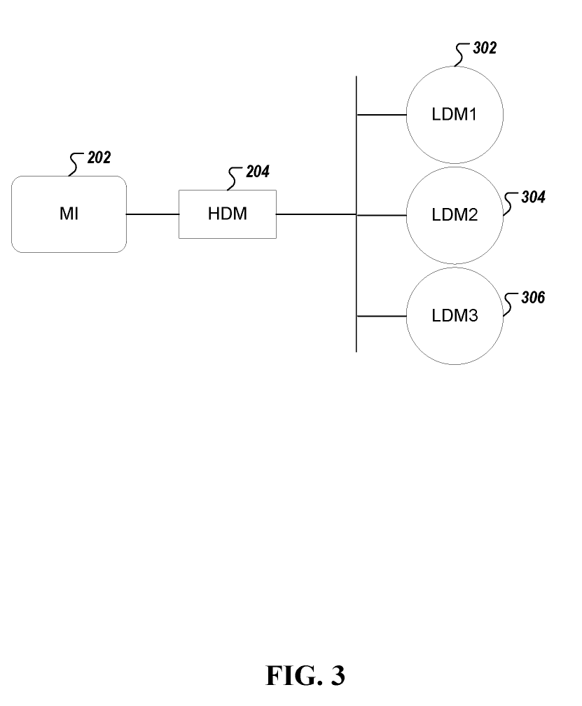

FIG. 3 illustrates various levels in accordance with the hierarchy defining mechanism. In some implementations, there is provided a first level defining mechanism (LDM 1302) configured to define a first level of activity in respect of a mapped item. A mapped item is described above.

Typically, this first level is a super node/level or a parent node/level. In response to a first tap, a first level is activated.

In at least one embodiment, these first level items can be linearly arranged. A primary action is displayed at this level with secondary actions in context menu.

In some implementations, there is provided a second level defining mechanism (LDM 2) 304 configured to define a second level of activity in respect of an object to be achieved and which is pursuant to at least one of the first levels of activity. A mapped item is described above.

Typically, this second level is a node/level or a child node/level. In response to a second tap, a second level is activated.

In at least one embodiment, in response to a first tap on an item at the first level, a user enters a corresponding second level. This level has all actionable items contextual to the first level tapped action item.

In some implementations, there is provided a third level defining mechanism (LDM 3) 306 configured to define a third level of activity in respect of an object to be achieved and which is pursuant to at least one of the second levels of activity. A mapped item is described above.

Typically, this third level is a sub node/level or a parent grandchild node/level. In response to a third tap, a third level is activated.

In at least one embodiment, in response to a second tap on an item at the second level, a user enters a corresponding third level. This level has all actionable items contextual to the second level tapped action item.

In some implementations, there is provided an intelligent action card (IAC) activated or authorized to carry out an action in relation to at least a mapped item (MI). In some implementations, the impactor is an intelligent action card. Depending upon a learned context, the intelligent action cards become context-oriented intelligent action cards. As per the mapped item(s), the intelligent action card includes at least one of an information item, an objective item, a decision item, an action item, a node item, or the like task-related items. In other words, these cards can be used for prescriptions, sharing, transmission, notifications, profiles, diagnosis, prognosis, examinations. These intelligent actions cards can be auto-generated and auto-presented at nodes in the healthcare system as defined by the electronic health record and practice management system and method. In some implementations, the impactor or the intelligent action card is formed at a second tap (second level) or a third tap (third level).

In some implementations, there is provided a customization mechanism configured to customize each item or task. Thus, a user, with rights, can modify arrangement of items or tasks in a given protocol. Modular plug-ins can be provided to facilitate this. This allows a user to customize what they want to see and what they don't. In other words, every information item is logically divided into micro-interfaces i.e. cards. These information interfaces can be plugged in or plugged out of the main interface based on user's need. This arrangement is part of customization mechanism.

The use of this system and method facilitates a design, which allows provisioning of an interface such that actions can continue seamlessly over a plurality of connected platforms, nodes, devices, and the like. There is uniformity of view and design, which makes use intuitive. This is called a one continuum, one design, one behavior technique and concept. From web to mobile to browser to wearables to watch, the system and method facilitates a similar look and facilitates a similar behavior. This helps in reducing a user's learning curve.

The technical advancement lies in achieving any actionable item in an electronic health record and practice management system or method in three taps or less. This is enabled by organizing all items across a domain in a specified manner with correlation(s) in the items. Since, practitioners or doctors have limited time in order to use an electronic health record and practice management system and method at a point of care, it is essential that an intuitive, predicable, hassle-free solution is provided which pre-empts a practitioner's or a doctor's next move based on previous data.

A user may provide user input through any suitable input device or input mechanism such as but not limited to a keyboard, a mouse, a joystick, a touchpad, a virtual keyboard, a virtual data entry user interface, a virtual dial pad, a software or a program, a scanner, a remote device, a microphone, a webcam, a camera, a fingerprint scanner, a cave, pointing stick

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

Although a few implementations have been described in detail above, other modifications are possible. For example, while a client application is described as accessing the delegate(s), in other implementations the delegate(s) may be employed by other applications implemented by one or more processors, such as an application executing on one or more servers. In addition, the logic flows depicted in the figures do not require the particular order shown, or sequential order, to achieve desirable results. In addition, other actions may be provided, or actions may be eliminated, from the described flows, and other components may be added to, or removed from, the described systems. Accordingly, other implementations are within the scope of the following claims.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A computer-implemented method performed by one or more processors, comprising:
identifying, by the one or more processors, an information item corresponding to a task in an electronic record and practice management environment;
identifying, by the one or more processors, an objective item corresponding to the task in the electronic record and practice management environment;
identifying, by the one or more processors, a decision item corresponding to the task in the electronic record and practice management environment;
identifying, by the one or more processors, an action item corresponding to the task in the electronic record and practice management environment;
identifying, by the one or more processors, a context item corresponding to the task in the electronic record and practice management environment;
in response to identifying the information item, the object item, the decision item, the action item, and the context item, displaying, by the one or more processors, an interactive mapping between the task and the information item, the object item, the decision item, the action item, and the context item on a display of a client device.

2. The computer-implemented method of claim 1, further comprising:
receiving, by the one or more processors, a first tap on the task on the display of the client device; and
in response to the receiving the first tap, displaying, by the one or more processors, the information item, the decision item, the action item, and the context item in a linear arrangement on the display of the client device.

3. The computer-implemented method of claim 2, further comprising:
receiving, by the one or more processors, a second tap on the task on the display of the client device; and
in response to receiving the second tap, displaying, by the one or more processors, first contextual information corresponding to the information item, the decision item, the action item, and the context item on the display of the client device.

4. The computer-implemented method of claim 3, further comprising:
receiving, by the one or more processors, a third tap on the task on the display of the client device; and
in response to receiving the third tap, displaying, by the one or more processors, second contextual information corresponding to the first contextual information on the display of the client device.

5. The computer-implemented method of claim 4, wherein the client device receives the first tap before the second tap, the first tap before the third tap, and the second tap before the third tap.

6. The computer-implemented method of claim 1, further comprising receiving, by the one or more processors, weights to assign to the information item, the decision item, the action item, and the context item.

7. The computer-implemented method of claim 1, wherein the task includes prescribing medicine to a patient from a doctor.

8. A system comprising:
one or more computers and one or more storage devices storing instructions that are operable, when executed by the one or more computers, to cause the one or more computers to perform operations comprising:
identifying, by one or more processors, an information item corresponding to a task in an electronic record and practice management environment;
identifying, by the one or more processors, an objective item corresponding to the task in the electronic record and practice management environment;
identifying, by the one or more processors, a decision item corresponding to the task in the electronic record and practice management environment;
identifying, by the one or more processors, an action item corresponding to the task in the electronic record and practice management environment;
identifying, by the one or more processors, a context item corresponding to the task in the electronic record and practice management environment;
in response to identifying the information item, the object item, the decision item, the action item, and the context item, displaying, by the one or more processors, an interactive mapping between the task and the information item, the object item, the decision item, the action item, and the context item on a display of a client device.

9. The system of claim 8, wherein operations further comprise:
receiving, by the one or more processors, a first tap on the task on the display of the client device; and
in response to the receiving the first tap, displaying, by the one or more processors, the information item, the decision item, the action item, and the context item in a linear arrangement on the display of the client device.

10. The system of claim 9, wherein operations further comprise:
receiving, by the one or more processors, a second tap on the task on the display of the client device; and
in response to receiving the second tap, displaying, by the one or more processors, first contextual information corresponding to the information item, the decision item, the action item, and the context item on the display of the client device.

11. The system of claim 10, wherein operations further comprise:
receiving, by the one or more processors, a third tap on the task on the display of the client device; and
in response to receiving the third tap, displaying, by the one or more processors, second contextual information corresponding to the first contextual information on the display of the client device.

12. The system of claim 11, wherein the client device receives the first tap before the second tap, the first tap before the third tap, and the second tap before the third tap.

13. The system of claim 8, wherein operations further comprise receiving, by the one or more processors, weights to assign to the information item, the decision item, the action item, and the context item.

14. The system of claim 8, wherein the task includes prescribing medicine to a patient from a doctor.

15. A non-transitory computer-readable medium storing software comprising instructions executable by one or more computers which, upon such execution, cause the one or more computers to perform operations comprising:
identifying, by one or more processors, an information item corresponding to a task in an electronic record and practice management environment;
identifying, by the one or more processors, an objective item corresponding to the task in the electronic record and practice management environment;
identifying, by the one or more processors, a decision item corresponding to the task in the electronic record and practice management environment;
identifying, by the one or more processors, an action item corresponding to the task in the electronic record and practice management environment;
identifying, by the one or more processors, a context item corresponding to the task in the electronic record and practice management environment;
in response to identifying the information item, the object item, the decision item, the action item, and the context item, displaying, by the one or more processors, an interactive mapping between the task and the information item, the object item, the decision item, the action item, and the context item on a display of a client device.

16. The computer-readable medium of claim 15, wherein operations further comprise:
receiving, by the one or more processors, a first tap on the task on the display of the client device; and
in response to the receiving the first tap, displaying, by the one or more processors, the information item, the decision item, the action item, and the context item in a linear arrangement on the display of the client device.

17. The computer-readable medium of claim 16, wherein operations further comprise:
receiving, by the one or more processors, a second tap on the task on the display of the client device; and
in response to receiving the second tap, displaying, by the one or more processors, first contextual information corresponding to the information item, the decision item, the action item, and the context item on the display of the client device.

18. The computer-readable medium of claim 17, wherein operations further comprise:
receiving, by the one or more processors, a third tap on the task on the display of the client device; and
in response to receiving the third tap, displaying, by the one or more processors, second contextual information corresponding to the first contextual information on the display of the client device.

19. The computer-readable medium of claim 18, wherein the client device receives the first tap before the second tap, the first tap before the third tap, and the second tap before the third tap.

20. The computer-readable medium of claim 15, wherein operations further comprise receiving, by the one or more processors, weights to assign to the information item, the decision item, the action item, and the context item.
